(19) United States
(12) Patent Application Publication
(10) Pub. No.: US 2003/0203963 A1
(43) Pub. Date: Oct. 30, 2003

(54) Antimicrobial agent

(76) Inventors: Dieter Elsser, Bargum (DE); Andrew John Morgan, Vedbaek (DK); Linda Valerie Thomas, Dorset (GB); Shukun Yu, Malmoe (SE)

Correspondence Address: FROMMER LAWRENCE & HAUG, 745 FIFTH AVENUE- 10TH FL., NEW YORK, NY, 10151, US

(21) Appl. No.: 10/396,003

(22) Filed: Mar. 25, 2003

Related U.S. Application Data
(63) Continuation-in-part of application No. PCT/GB01/04328, filed on Sep. 27, 2001.

(30) Foreign Application Priority Data
Apr. 4, 2002 (WO) 02/26060
Sep. 27, 2002 (GB) 0023687.7
Sep. 27, 2002 (GB) 0023686.9

(51) Int. Cl. A61K 31/35

(52) U.S. Cl. 514/460

(57) ABSTRACT

The present invention provides an antimicrobial composition for use against a micro-organism selected for Listeria, Salmonella, Bacillus, Saccharomyces, Pseudomonas, Clostridium, Lcatobacillus, Brochothrix, Micrococcus, Yersinia, Enterobacter and Zygosaccharomyces, said composition comprising a cyclic compound having Formula (I), or a derivative thereof, wherein R¹ and R² are independently selected from —OH, ═O, and OR′, wherein R′ is H or —COR″, and R″ is C₁₋₁₀ alkyl; wherein R³ is a substituent comprising an OH-group, wherein R⁴ and R⁵ are each independently selected from a hydrocarbyl group, H, OH or ═O, or represent a bond with an adjacent atom on the ring of the cyclic compound. The invention further relates to a process preventing and/or inhibiting the growth of, and/or killing, micro-organisms in a material, and the use of a cyclic compound having Formula (I).

### REFERENCE TO RELATED APPLICATIONS/INCORPORATION BY REFERENCE

This application is a Continuation-in-Part of PCT/GB01/04328, filed on Sep. 27, 2001, designating the U.S., published on Apr. 4, 2002 as WO 02/26060 A1 and claiming priority from GB 0023687.7 filed on Sep. 27, 2000 and GB 0023686.9 filed on Sep. 27, 2000. All of the above-mentioned applications, as well as all documents cited herein and documents referenced or cited in documents cited herein, are hereby incorporated by reference.

The present invention relates to antimicrobial agents. More specifically, the invention relates to the antimicrobial activity of a series of anhydrofructose derivatives.

Food degradation from various sources is recognised in the literature and individual chemicals are known which will inhibit one aspect or another of degradation derived from a single source. Degradation, and the loss of colour or flavour of freshly cut plant parts are known to be caused by oxidation, enzymes, microbes, and metal ions. For example, acidulants are known to prevent microbial degradation by maintaining a relatively low pH environment but their effectiveness is only temporary.

Salmonella, of which there are over two thousand different strains, is one of the major causes of food poisoning in humans. Salmonella is a genus of rod-shaped Gram-negative Enterobacteriaceae that inhabit the intestine and cause infections such as gastroenteritis and typhoid. If invasive, they can cause enteric fevers (for example, typhoid caused by Salmonella typhi, or paratyphoid fever caused by Salmonella paratyphi). Other strains of Salmonella are associated with food poisoning. (usually Salmonella Typhimurium, Salmonella Panama or Salmonella Enteritidis, the latter notorious for the contamination of poultry) and occasionally septicaemia in non-intestinal tissues.

It is well known in the art that Salmonella cannot propagate at pH values below 4.5. As a consequence, mildly acid products such as fine food and non-fermented meat products are especially susceptible to attack by Salmonella.

For meat products, nitrite is often used as a preservative. However, the addition of nitrite is restricted for toxicological reasons (due to its acute toxicity, together with the dangers associated with nitrosamine formation). As a result, Salmonella is only inhibited at concentrations of nitrite beyond 1,000 ppm, which are far beyond legal limits.

Instead, it has been shown that combinations of nitrite and sorbic acid can increase the effectiveness against Salmonella [Inhibition of Salmonella by Sodium Nitrite and Potassium Sorbate in Frankfurters, Journal of Food Science, 47, 1982, p. 1615 ft]. Inhibition has been observed at concentrations beyond 50 ppm of nitrite combined with 2600 ppm sorbic acid.

Other agents such as bacteriocins (Nisin) are unable to inhibit Salmonella in food, whereas benzoic acid is unsuitable because the inhibitory effect can only be observed in acid products. The inhibitory effect of phytogenic ingredients (or “natural substances”) such as oil extracts from different spices, has also been tested, but again the concentrations required for achieving the inhibitory effect on Salmonella were too high and the sensorical influence on the food was too strong.

Thus, to date, the use of chemical substances has been severely limited because on the one hand they have to be safe from a toxicological view point, but on the other hand they must not influence the product sensorically.

The present invention seeks to alleviate the problems associated with prior art chemical substances and to provide new antimicrobial compositions based on anhydrofructose derivatives. In particular, the invention seeks to provide antimicrobial agents that are suitable for use in foodstuffs/feed.

In a first aspect, the invention provides an antimicrobial composition for use against a micro-organism selected from Listeria, Salmonella, Bacillus, Saccharomyces, Pseudomonas, Clostridium Lactobacillus, Brochothrix, Micrococcus, Yersinia, Enterobacter and Zygosaccharomyces, said composition comprising a cyclic compound having Formula I,

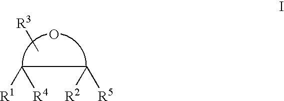

or a derivative thereof,

wherein R¹ and R² are independently selected from —OH, ═O, and OR′, wherein R′ is H or —COR″, and R″ is C₁₋₁₀ alkyl;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound.

In a second aspect, the invention provides a process for preventing and/or inhibiting the growth of, and/or killing, micro-organisms in a material, the process comprising the step of contacting the material with a cyclic compound having Formula I,

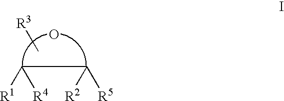

or a derivative thereof,

wherein R¹ and R² are independently selected from —OH, ═O, and OR′, wherein R′ is H or —COR″, and R″ is C₁₋₁₀ alkyl;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound;

wherein said micro-organism is selected from Listeria, Salmonella, Bacillus, Saccharomyces, Pseudomonas, Clostridium, Lactobacillus, Brochothrix, Micrococcus, Yersinia, Enterobacter and Zygosaccharomyces.

In a third aspect, the invention relates to the use of a compound having Formula I or a derivative thereof,

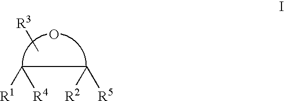

wherein R¹ and R² are independently selected from —OH, ═O, and OR′, wherein R′ is H or —COR″, and R″ is C₁₋₁₀ alkyl;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound;

for preventing and/or inhibiting the growth of, and/or killing, micro-organisms in a material, wherein said micro-organism is selected from Listeria, Salmonella, Bacillus, Saccharomyces, Pseudomonas, Clostridium Lactobacillus, Brochothrix, Micrococcus, Yersinia, Enterobacter and Zygosaccharomyces.

Preferably, the material is a foodstuff or feed. Thus, in a preferred aspect, the present invention relates to antimicrobial substances that are suitable for use in foodstuffs and/or feed to inhibit food poisoning and spoiling bacteria contained therein.

By way of definition, the term “antimicrobial” refers to a substance that kills or prevents or inhibits the growth or reproduction of micro-organisms. Antimicrobials are generally classified according to the type of micro-organism they are effective against. For example, antibacterial substances are effective against bacteria, antifungal substances are effective against fungi, including yeast, and antiviral substances are effective against viruses. Certain antimicrobials can be used internally, for example antibiotic medications, whereas other antimicrobials are for external use only, such as antiseptics.

In a preferred aspect, the cyclic compound of the invention is a compound having formula I,

or a derivative thereof,

wherein R¹ and le are independently selected from —OH, ═O;

wherein R³ is a substituent comprising an —OH group;

wherein R⁴ and R⁵ are each independently selected from a hydrocarbyl group, H, OH or ═O,

or represent a bond with an adjacent atom on the ring of the cyclic compound.

In a more preferred aspect, the cyclic compound of the invention is a compound having Formula II

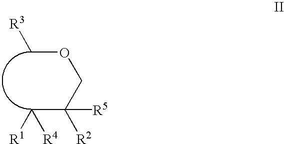

or a derivative thereof, wherein R¹, R², R³, R⁴, and R⁵ are as defined above.

In a further preferred aspect, the cyclic compound of the invention is a compound having Formula III

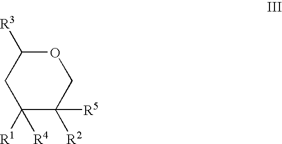

or a derivative thereof; wherein R¹, R², R³, R⁴, and R⁵ are as defined above.

In one preferred aspect of the invention the groups R⁴ and R⁵ of the general formula may independently be a hydrocarbyl group.

The term “hydrocarbyl group” as used herein means a group comprising at least C and H and may optionally comprise one or more other suitable substituents. Examples of such substituents may include halo-, alkoxy-, nitro-, hydroxy, carboxyl, epoxy, acrylic, hydrocarbon, N-acyl, or cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen.

The groups R⁴ and R⁵ of the general formula may independently be selected from alkyl, alkenyl, cycloalkyl and aryl or may together represent an alkylene.

In a particularly preferred aspect of the invention, the derivative of the compound of Formula I is an ester. The term “ester” includes mono-, di-, tri- and poly-esters.

Preferably, the derivative of the compound of formula I is an ester wherein an ester linkage is formed from the —OH group of the R³ substituent. In this aspect preferably the derivatised R³ substituent is a group of the formula —(CH₂)ₙ—OC(O)—(CH₂)ₚCH₃, wherein n and p are independently of each other from 1 to 24, preferably from 1 to 20, preferably from 1 to 10, preferably from 1 to 5, or preferably 1, 2, or 3. In yet a further preferred embodiment the derivatised R³ substituent is a group of the formula —CH₂—OC(O)—(CH₂)ₚCH₃, wherein p is from 1 to 24, preferably from 1 to 20, or p is from 1 to 10, or p is from 1 to 5, and n=1, 2, or 3.

Preferably, the derivative of the compound of formula I is an ester wherein the R¹ substituent and/or the R² substituent is an —OH group and wherein an ester linkage is formed from the —OH group of the R¹ substituent and/or the R² substituent. In this aspect preferably the derivatised R¹ substituent and/or the R² substituent is a group of the formula —(CH₂)ₙ—OC(O)—(CH₂)ₚCH₃, wherein n and p are independently of each other from 1 to 24, preferably from 1 to 20, preferably from 1 to 10, preferably from 1 to 5, or preferably 1, 2, or 3. In yet a further preferred embodiment the derivatised R¹ substituent and/or the R² substituent is a group of the formula —CH₂—OC(O)—(CH₂)ₚCH₃, wherein p is from 1 to 24, preferably from 1 to 20, or p is from 1 to 10, or p is from 1 to 5, and n=1, 2, or 3.

Preferably, the derivative of the compound of formula I is an ester wherein the R¹ substituent and/or the R² substituent is an —OH group and wherein an ester linkage is formed from the —OH group of the R¹ substituent and/or the R² substituent. In this aspect preferably the derivatised R¹ substituent and/or the R² substituent is a group of the formula —OC(O)—(CH₂)ₚCH₃, wherein p is from 1 to 24, preferably from 1 to 20, preferably from 1 to 10, preferably from 1 to 5, or preferably 1, 2, or 3.

In a preferred aspect the compound of formula I is a diester wherein the R¹ substituent is an —OH group and wherein the ester linkages are formed from the —OH group of the R⁴ substituent and from the —OH group of the R³ substituent.

In a highly preferred aspect a derivative of the compound of formula I is a compound of the formula

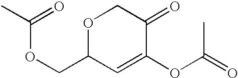

This compound (3,6-di-O-acetyl-1,5-anhydro-4-deoxy-D-glycero-hex-3-enopyranose-2-ulose) may be prepared in accordance with the teaching of Andersen et al. (1998), Structure of 1,5-anhydro-D-fructose: X-ray analysis of crystalline acetylated dimeric forms, J. Carbohydr. Chem. 17: 1027-1035.

The aspect of the present invention wherein the derivative of the compound of formula I is an ester is particularly preferred because the compound may be lipophilic and/or may have both hydrophobic and hydrophilic properties. When the compound has both hydrophobic and hydrophilic properties the compound readily resides at a water/oil interface of an emulsion.

The residence of the compound at a water/oil interface of an emulsion may allow it to act as an emulsifier. Thus the present invention may further provide compounds having a dual functional effect. The compounds may act both as an antimicrobial and as an emulsifier.

In particularly preferred aspect of the invention, the cyclic compound is selected from Ascopyrone P, Ascopyrone M, Ascopyrone T, Ascopyrone T₁, Ascopyrone T₂, and Ascopyrone T₃, and mixtures thereof, the structures of which are shown below.

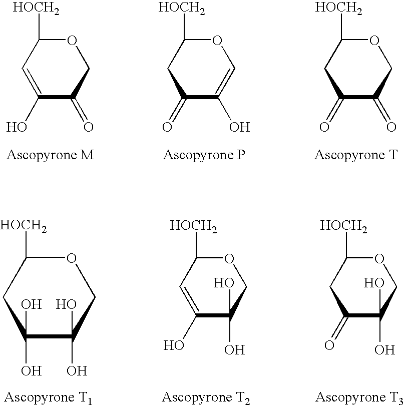

Ascopyrone is a known compound. In 1978 and 1981, a group of American scientists prepared Ascopyrone P by pyrolysis of amylopectin, amylose and cellulose at the Wood Chemistry laboratory in Montana, with the intention of using Ascopyrone P as a stating material for organic synthesis [Shafizadeh, F., Furneaux R-H., Stevenson, T. T., and Cochran, T. G., 1,5-Anhydro-4-deoxy-D-glycero-hex-1-en-3-ulose and other pyrolysis products of cellulose, Carbohydr. Res. 67(1978): 433-447; Stevenson, T. T., Stenkmap, R. E., Jensen, L. H., Cochran, T. T., Shafizadeh, F., and Furneaux R. H., The crystal structure of 1,5-anhydro-4-deoxy-D-glycero-hex-1-en-3-ulose, Carbohydr. Res. 90(1981): 319-325]. They characterised Ascopyrone P by, for example, ¹H and ¹³C NMR, and IR spectroscopy techniques. A 3-dimensional structure of Ascopyrone P was provided. The yield of Ascopyrone P obtained by pyrolysis was under 3% and complicated separation methods had to be used.

The natural occurrence of Ascopyrone P in some species of very scarcely studied fungi collected from the Alps has been taught [M.-A. Baute, G. Deffieux, J. Vercauteren, R Baute, and Badoc A., Enzymatic activity degrading 1,4-α-glucans to Ascopyrones P and T in Pezizales ad Tuberales, Phytochemistry, 33 (1993): 4145]. The occurrence of Ascopyrone P in fungi immediately prompted the hypothesis that Ascopyrone P would act as an antibiotic. However, Ascopyrone P did not function satisfactorily as an antibiotic in the disclosed tests.

Many of the compounds of the present invention can be derived from 1,5-anhydrofructose. 1,5-Anhydrofructose is monoketo sugar found in bacteria, red algae, fungi and mammals. In red algae and fungi 1,5-anhydrofructose is produced by the action of o-1,4-glucan lyase [EC 4.2.2.13] from floridean starch and glycogen, respectively.

When the compound of the present invention is prepared from 1,5-anhydro-D-fructose, preferably the 1,5-anhydro-D-fructose is prepared in accordance with GB-A-2296717. In other words, preferably the 1,5-anhydro-D-fructose is prepared by a method comprising treating an α-1,4-glucan with the enzyme α-1,4-glucan lyase characterised in that enzyme is used in substantially pure form.

Ascopyrone P and Ascopyrone T can be produced enzymatically from 1,5-anhydro-D-fructose using cell-free extract prepared from the fungi of the order Pezizales, such as Plicaria leiocarpa and Anthracobia melaloma, and the order of Tuberales, such as, Tuber melanosporum. Ascopyrone T₁ is the dihydrate form of Ascopyrone T, whereas Ascopyrone T₂ and T₃ are the tautomeric monohydrate forms of Ascopyrone T.

Ascopyrone M can be produced from 1,5-anhydro-D-fructose by EDTA-sensitive dehydratases isolated from the fungi Morels, such as Morchella vulgaris, Gyromitres, pezizes, such as Peziza echinospora.

Ascopyrone M, P and T can also be produced chemically by treating 1,5-anhydro-D-fructose with alkali under mild conditions [Studies on the degradation of some pentoses and of 1,5-anhydro-D-fructose, the product of the starch-degrading enzyme α-1,4-glucan lyase; Thesis, Ahmad, T., The Swedish University of Agricultural Sciences, Sweden, 1995].

When the compound of the present invention is prepared by chemical means, it may be prepared in accordance with one of the following methods:

(1) Ascopyrone P may be produced by treating 1,5-anhydro-D-fructose with non-aqueous acid at elevated temperature, for example at 70° C.

(2) Ascopyrones (for example, Ascopyrone P, T and M) may be produced from 1,5-anhydro-D-fructose by alkaline treatment according to Ahmad, T., 1995.

The structures of all ascopyrones produced were confirmed by NMR techniques.

Preferably, the compound of the present invention is prepared by enzymatic means as disclosed in M.-A. Baute et al, [Phytochemistry, 33 (1993): 4145). For example ascopyrones (such as, Ascopyrone P, T and M) may be produced from 1,5-anhydro-D-fructose using enzymatic methods as disclosed in M.-A. Baute et al.

In another preferred aspect, the cyclic compound of the invention is of Formula IV,

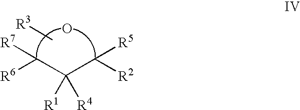

or a derivative thereof,

wherein R¹ and R² are independently selected from —OH, ═O, and OR′, wherein R′ is H or —COR″, and R″ is C₁₋₁₀ alkyl;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound;

wherein R⁶ and R⁷ are each independently selected from H, OH or ═O, or represent a bond with an adjacent atom on the ring of the cyclic compound.

In a more preferred aspect, the cyclic compound of the invention is of formula V,

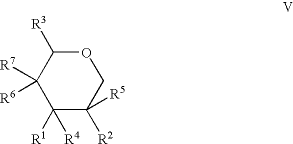

or a derivative thereof, wherein R¹, R², R³, R⁴, R⁵, R⁶ and R⁷ are as defined above.

Even more preferably, the cyclic compound of the invention is selected from one or more of the following:

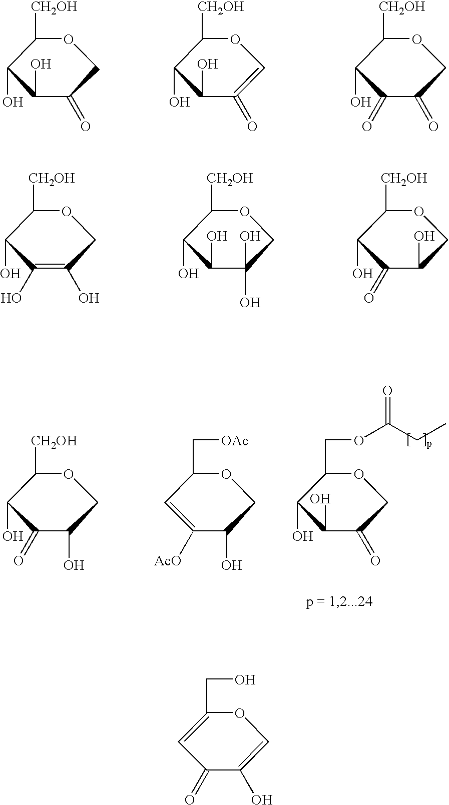

In a particularly preferred aspect of the invention, R³ is or comprises a CH₂OH group.

Preferably, the cyclic compound of the invention comprises a five or a six membered ring.

In a particularly preferred embodiment of the invention, the cyclic compound having Formula I has an antimicrobial effect against a micro-organism selected from Listeria monocytogenes, Listeria innocua, Salmonella Typhimurium, Salmonella sp., Bacillus cereus, Bacillus subtilis, Saccharomyces cerevisiae, Saccharomyces cerevisiae var. paradoxus, Saccharomyces carlsbergensis Pseudomonas fluorescens, Clostridium sporogenes, Lactobacillus sake, Brochothrix thermosphacta Micrococcus luteus, Yersinia enterocolitica, Enterobacter aerogenes, Zygosaccharomyces bailii.

The cyclic compound of the invention may be used alone, or in combination with other components, for example, one or more chelators (such as EDTA sodium salt, polyphosphate or citrate) and/or one or more antioxidants (such as ascorbate, isoascorbate, ascorbate palmitate, BHA or BHT).

Tests indicate that APP is stable in water at 24° C. for 2 weeks, but completely disappears after 2 months. It has been reported that APP is stable at pH 1.5-5.5, and less stable at increasing pH. At pH 11-12.5 APP has a half-life of 5 h. Since most food is acidic or neutral, the stability may therefore be improved by using APP in combination with an antioxidant such as those listed hereinbefore.

In one preferred embodiment, the compound is used in combination with one or more preservatives. By way of definition, in the broadest sense, the term “preservative” is intended to encompass all substances which inhibit the development of, or kill, micro-organisms. In a narrower sense, it is generally understood that preservatives are used in concentrations of 0.5% or less. Food additives which are allowed to be used as preservatives are listed in the Regulation No. 95/2/EG of the European Parliament and Council of Feb. 20, 1995, relating to food additives other than colouring agents and sweeteners.

Typical food preservatives permitted in the EU which are suitable for use in combination with the compounds of the invention include sorbic acid, benzoic acid, PHB ester (p-hydroxybenzoate), and sulphur dioxide. The mode of action of these preservatives, together with their range of effects are listed below.

Sorbic Acid (E200 to 203):

Mode of action: inhibits different enzymes in the cells of the micro-organisms.

Range of effects: mainly against yeasts and moulds as well as catalase-positive bacteria. Catalase-negative bacteria as well as lactic acid bacteria and clostridia are not inhibited.

Effective concentration: 500-3000 ppm.

Permitted maximum quantities in food: up to 2000 ppm in potato dough, processed cheese, packed bread, fine bakery products, emulsified sauces etc.

Benzoic Acid (E210 to 213):

Mode of action: inhibits exchange of oxygen through the cellular membrane and affects the enzymatic structure.

Range of effects: for acid products only, up to approx. pH 4.5; inhibits yeasts and moulds, restricted inhibition of bacteria (no, or only very little, inhibition of lactic acid bacteria and clostridia).

Permitted maximum quantities in food: 500 ppm in aspic, fruit preparations, marmalades etc.

PHB Ester (p-hydroxybenzoate) (E214 to 219)

Mode of action: damages the bacterial membrane because of the surface activity, poisonous to protoplasm because of protein denaturation.

Range of effects: mainly inhibits yeasts and fungi, but also Gram-positive bacteria in a pH range between 3.0 and 8.0.

Effective concentration: sensorical influence at concentrations beyond approx. 0.08%.

Sulphur Dioxide (E220 to 224; E 226 to 227)

Mode of action: depends on pH to a great extent, in practice it is only effective at acidic pH values (<4.0). Very complex mechanisms.

Range of effects: mainly antibacterial, above all against Gram-negative, aerobic bacteria.

Effective concentrations: 250-500 ppm for inhibition of aerobic, Gram-negative bacteria, 800-2000 ppm against Gram-positive bacteria, yeasts, and moulds.

Permitted maximum quantity in food products: max. 2000 ppm in dry fruits, grape juice concentrate for home production of wine, in some cases only max. quantities of 20-30 ppm are permitted.

For more specific applications, the compounds of the present invention may also be used in combination with the following preservatives: biphenyl, diphenyl, orthophenylphenol, thiabendazol, nisin, natamycin, hexamethylentetramine, dimethyldicarbonate, boric acid, sodiumtetraborate, nitrite, propionic acid and propionate, and lysozyme. The mode of action of these preservatives, together with their range of effects and specific uses are listed below.

Biphenyl, Diphenyl (E 230)

Range of effects: Inhibition of moulds.

Substance for treatment of fruits: surface treatment of citrus fruits.

Permitted maximum quantity: 70 ppm

Orthophenylphenol (E 231/E 232)

As with E230, limited to treatment of fruits as a surface treatment for citrus fruits.

Thiabendazol (E 233)

Surface treatment of citrus fruits and bananas.

Nisin (E 234)

Mode of action: Disturbance of membrane functions.

Range of effects: Gram-positive bacteria, no influence on Gram-negative bacteria

Permitted maximum quantity in food products (EL): 3 ppm in semolina pudding and similar products, 12.5 ppm (=12.5 IU/g) in ripened cheese and processed cheese, 10 ppm in clotted cream, 10 ppm in mascarpone.

Natamycin (Pimaricin (E235)

Mode of action: specifically attacks cell membrane, where—in general—an interaction with sterines occurs which increases the permeability of the membrane.

Range of effects: Moulds and yeasts, not effective against bacteria. Usual dosage rates are below approx. 50 mg/l. Maximum level is 1 mg/dm² on the surface, with a maximum penetration of 5 mm.

Applications: surface treatment of hard, semi-hard and semi-soft cheese and of dried, cured sausages.

Hexamethylentetramine (E 239)

Hexamethylentetramine is formed by adding ammonia to formaldehyde in an aqueous solution. The microbicidal effect is due to the formaldehyde.

Permitted only for Provolone cheese (25 ppm residual quantity).

Dimethyldicarbonate (E 242)

Permitted only for non-alcoholic drinks, non-alcoholic wine, and liquid concentrate.

Boric Acid, Sodiumtetraborate (E284/E 285)

Permitted only for caviar.

Nitrite (249 and E 250)

Permitted in the form of nitrite curing salt for treatment of meat products (“red products”). For cured and dried meat products which are not heat treated and for other cured meat products an addition of 150 ppm has been fixed as a guideline. These concentrations do not show a preservative effect. They are mainly added for their technological properties (formation of colour, taste) as well as for their antioxidant effects.

Propionic Acid and Propionate (E 280, E 281, E 282, and E 283)

Mode of action: similar to sorbic acid, pH<4.5 is optimal.

Accumulation in the cell leads to inhibition of enzymes.

Range of inhibition: moulds are inhibited at an pH of 5.5 by concentrations of 125 to 12500 ppm, for inhibition of bacteria higher concentrations are necessary (>16000 ppm).

Application: Sliced and packaged bread.

Permitted maximum quantity: 3000 ppm.

Lysozyme (E 1105)

Permitted only for ripened cheese.

Permitted maximum quantity: quantum satis.

Studies by the applicant of the inhibitive effects of the present compounds have been tested in a medium (Elliker broth) with an almost neutral pH (pH 6.8) and have been shown to be effective against both Gram-positive and Gram-negative bacteria. As many of the preservatives described above show an inhibitory effect mainly at low pH, the use of the compounds of the present invention clearly broadens the potential range of applications.

In principle, the use of substances for chemical preservation depends on the following factors:

(a) Toxicological harmlessness

the effects of the substance when applied acutely, subchronically, and for a long term period.

Testing of acute toxicity (LD₅₀), cinetics and metabolism, pharmacological effects, genotoxicity, etc.

(b) Technological/food chemical aspects:

Solubility in water: as growth takes place in the aqueous phase, a preservative has to be water-soluble

Reaction with food ingredients, problem of off-flavours (sensory acceptance)

Interferences with food ingredients (e.g. destruction of vitamin B1 by sulphuric acid)

The antimicrobial effectiveness of chemical substances in food and feed products is thus determined by a range of different factors. Among others, the composition of the population of micro-organisms, the composition of the food product (ingredients, pH, water activity, content of salt, etc.), the packaging, time-temperature-conditions, etc. are key factors that influence the inhibitory activities of the antimicrobial agent

The invention will now be described only by way of example, and with reference to the accompanying figures, wherein:

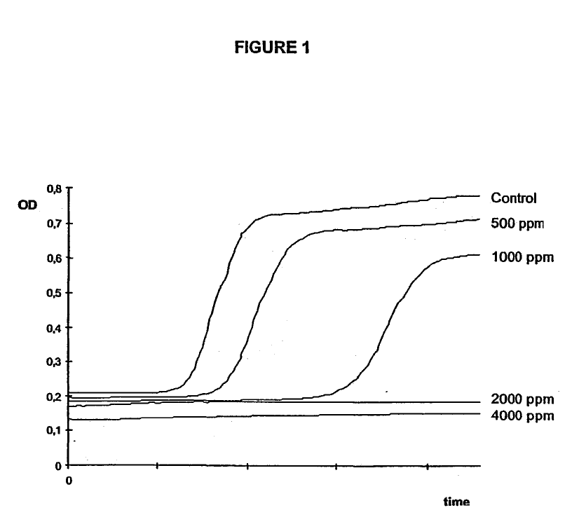

FIG. 1 shows the antimicrobial activity of Ascopyrone P (APP) against Salmonella Typhimurium DSMZ 554 (10e3 cfu/ml).

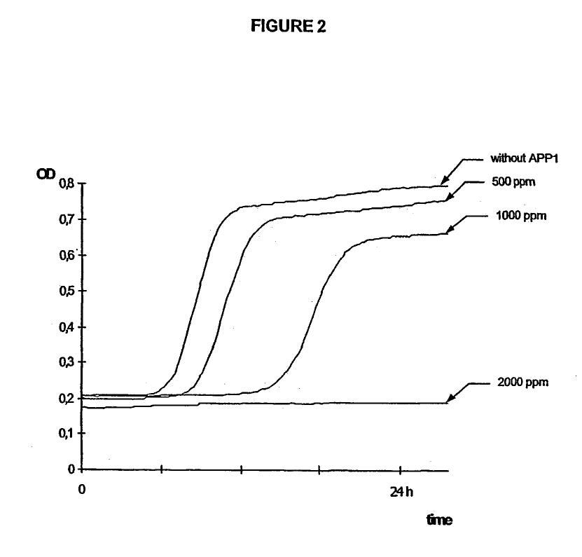

FIG. 2 shows the antimicrobial activity of Ascopyrone P (APP) against Salmonella Typhimurium DSMZ 554 (10e5 cfu/ml).

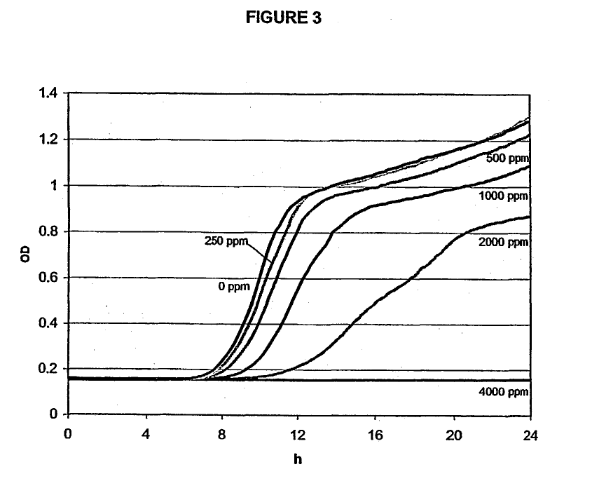

FIG. 3 shows the effect of APP against S. Typhimurium S29 at 30° C. over a period of 24 h.

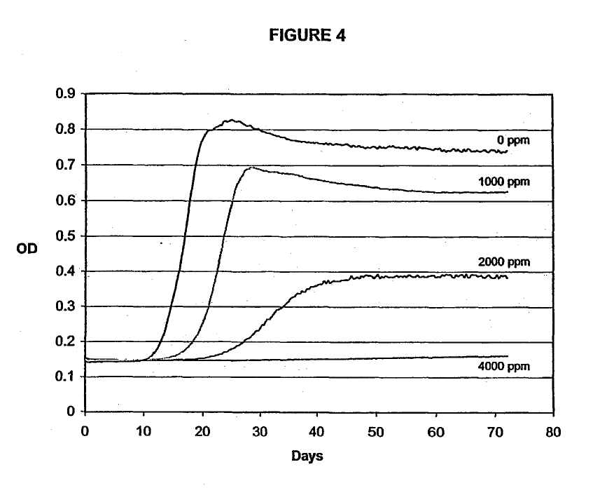

FIG. 4 shows the effect of APP on the growth of Br. thermosphacta CRA7883 over a 72 h period.

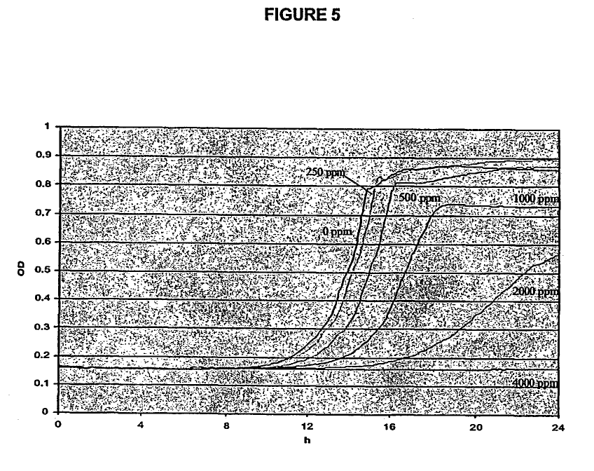

FIG. 5 shows the effect of APP on the growth of L. monocytogenes 272 at 30° C. over a period of 24 h.

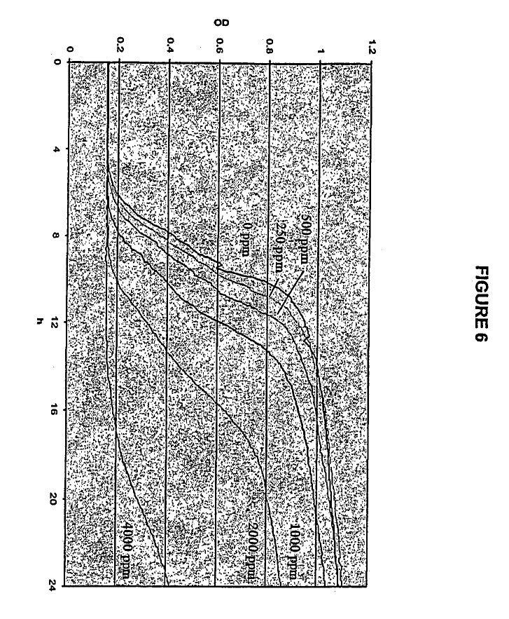

FIG. 6 shows the effect of APP on the growth of B. cereus 204 at 30° C. over a period of 24 h.

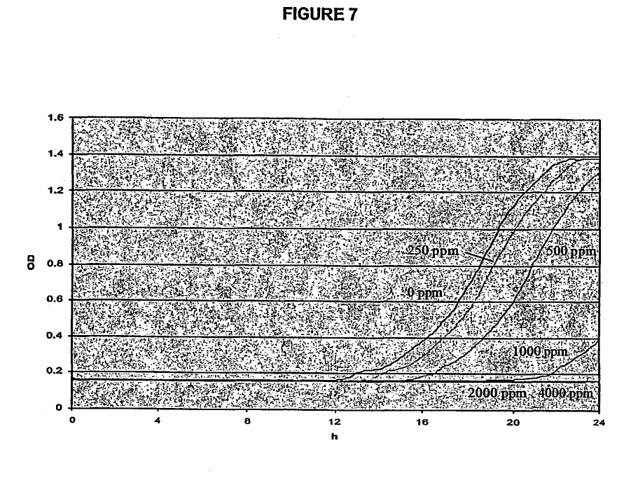

FIG. 7 shows the effect of APP on the growth of Ps. fluorescens 3756 at 30° C. over a period of 24 h.

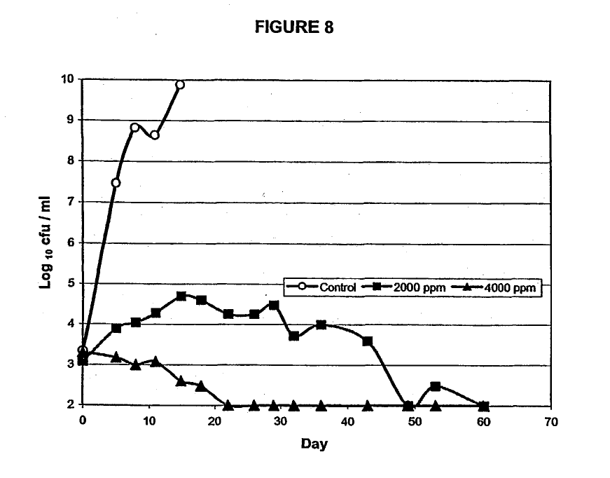

FIG. 8 shows the effect of APP on L. monocytogenes 272 in chicken soup at 8° C. (detection limit 10² cfu/g) over a 60 day period.

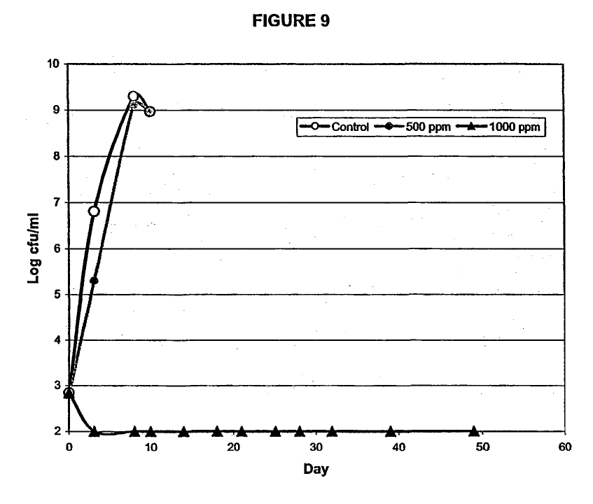

FIG. 9 shows the effect of APP against Ps. fluorescens 3756 in chicken soup at 8° C. (detection limit 10² cfu/g) over a 49 day period.

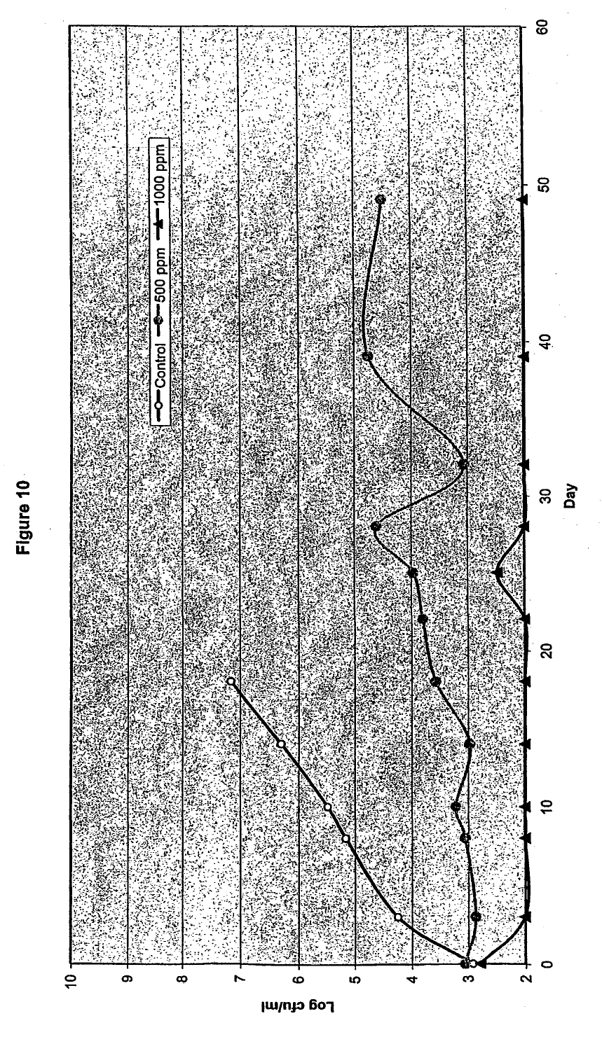

FIG. 10 shows the effect of APP against Salmonella Typhimurium S29 in chicken soup at 8° C. Detection limit 102 cfu/ml. Trial finished at 49 d

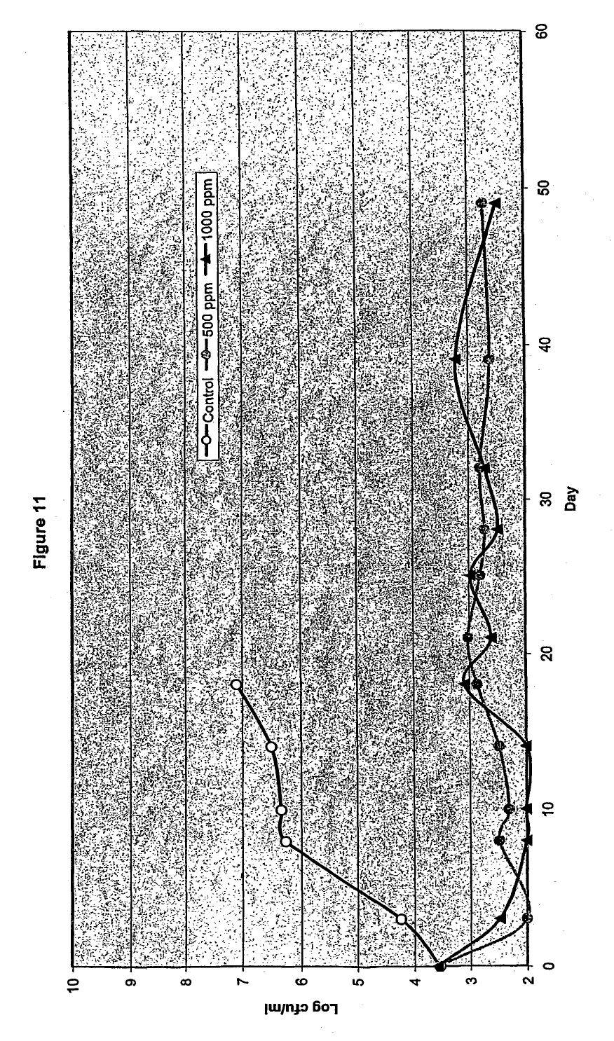

FIG. 11 shows the effect of APP against B. cereus 204 spores in chicken soup at 8° C. Detection limit 102 cfu/ml. Trial finished at 49 d

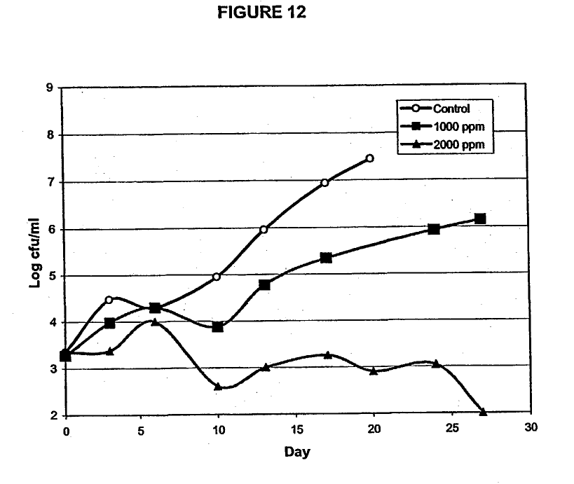

FIG. 12 shows the effect of APP on S. Typhimurium S29 in a cooked mince beef slurry system at 8° C.

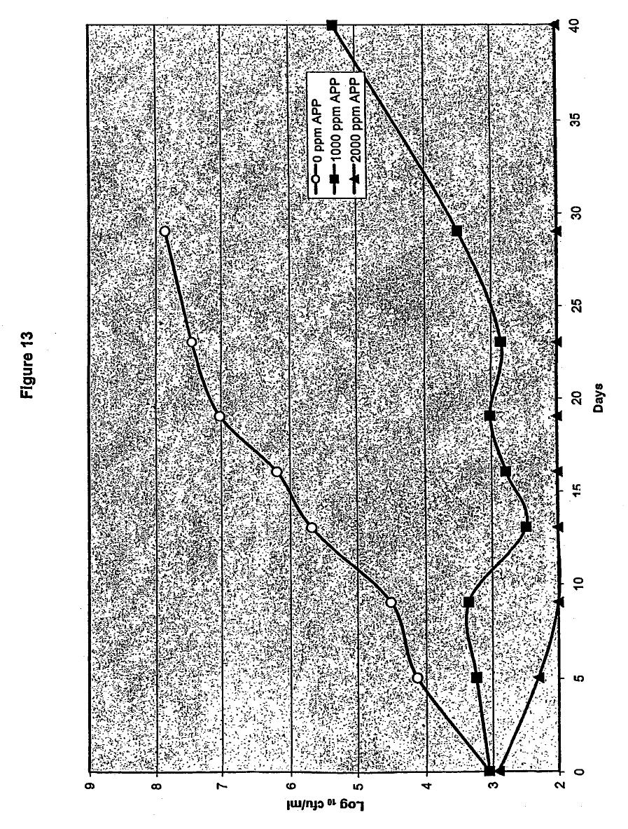

FIG. 13 shows the effect of APP against Salmonella Typhimurium S29 in minced chicken slurry at 8° C.

### EXAMPLES

1. Preliminary Investigation

Inhibition of Gram-Positive and Gram-Negative Bacteria

Antimicrobial activity was investigated using Salmonella Typhimurium and Listeria innocua.

Bacterial growth was carried at 37° C. for 24 hour for S. Typhimurium and 20 hours for Listeria innocua, both at an inoculation size of 10e3 or 10e5 cfu/ml (10³ or 10⁵ colony forming units per ml).

In both cases, APP showed a satisfactory inhibitory effect.

S. Typhimurium

For S. Typhimurium at 10e3 and 10e5, complete inhibition was observed at 2000 μg/ml and 4000 μg/ml (see FIGS. 1 and 2). Intermediate inhibition was observed at 500 μg/ml, with strong inhibition observed at 1000 μg/ml.

L. innocua

For L. innocua at 10e3, almost complete inhibition was observed at 2000 μg/ml, intermediate inhibition at 1000 μg/ml, and slight inhibition at 500 μg/mL At 10e5, intermediate inhibition was seen at 2000 μg/ml, slight inhibition at 1000 μg/ml, and almost no inhibition at 500 μg/mL

The tests described above were all performed under the optimal growth conditions for the bacteria at high inoculation levels. In food, the growth conditions for these bacteria will be less favourable and the inoculation level will be lower, therefore lower dosages will be needed to inhibit their growth. In other words, lower concentrations of the compounds of the invention will be necessary in food products, for a sufficient inhibition of food poisoning or food spoiling micro-organisms.

The above results clearly show that APP inhibits both Gram-negative bacteria of the Enterobacteriaceae type (Salmonella Typhimurium) and Gram-positive bacteria (Listeria innocua).

Further trials were undertaken to investigate the antimicrobial efficacy of ascopyrone P (APP) in laboratory media and in a range of food systems.

2. Materials and Methods

2.1 Test Strains

All micro-organisms were taken from storage at −80° C. Most organisms were tested as vegetative cell suspensions from overnight broth culture. Bacillus and Clostridium species were tested as endospore suspensions prepared earlier and stored at 4° C.

For broth cultures and Bioscreen testing most bacteria were grown in Brain Heart Infusion (BHI, Oxoid, pH 7.4). Lactobacillus sake A10 was grown in de Man, Rogosa, Sharpe medium (US, Oxoid). Yeasts were grown in Sabouraud Liquid medium (SLM, Oxoid). Most bacteria were cultured at 30° C. Lactic acid bacteria were grown on solid medium in enriched CO₂ atmosphere. Clostridium species were grown in Reinforced Clostridial Medium (RCM) at 37° C. anaerobically. Brochothrix thermosphacta fungi and yeasts were grown at 25° C. Fungi were cultured on Malt Extract Agar (MEA, Oxoid).

2.2 Ascopyrone P (APP) Samples

Sample E002012 was a dry powder that was dissolved in sterile de-ionised water. It was tested without filtration and used in the mini well diffusion test, initial Bioscreen runs (BS1211200; BS131200), and mini cidal experiment. The concentration of this sample was 49.3 mg/ml. Further APP samples were prepared as follows: 3.84 g of batch number APP20010213, as 5×4 ml volumes, was made up to a concentration of 169 mg APP/ml, and 5.5 g of batch number APP20010215, as 4×10 ml volumes, was made up to a concentration of 138 mg APP/ml. All samples were kept at −20° C. until use. Stock solutions were made up in de-ionised water and filter sterilised. These samples were used in Bioscreen run BS010411 and BS010420. APP20010213 was used in the following trials: chicken soup at 8° C. (TCS010412; TCS 010430); raw meat (TRM010521). APP20010215 was used in Bioscreen run BS010510, Clostridium sensitivity tests and in the following trials: chicken soup at 8° C. (TCS010521); apple juice at 25° C. (TAJ010530); milk (TMBG010605), sausage meat slurry test (TSM010605); further raw meat tests (TRM010620) and cooked beef and chicken slurry tests (TCM010619; TCC010704).

2.3 In vitro Growth Inhibition Test Methods Testing

2.3.1 Well Diffusion Testing

Seeded 10 ml agar plates of various test organisms were prepared, with inoculation of either 20 μl spore suspension or overnight broth. This gave an inoculum level of ca. 10⁵-10⁶ cfu/ml. After the plates had set, small wells were cut, and 20 μl of APP sample was loaded. Plates were incubated overnight at appropriate temperature and examined after 1-2 days (after which microbial growth is clearly visible) for zones of inhibition.

2.3.2 Bioscreen Testing

An automated Microbiology Reader Bioscreen C was used to measure growth curves of the strains in the presence and absence of APP. The Bioscreen C measures the development of turbidity (i.e. growth) kinetically by vertical photometry in 200 wells of a honeycomb microtitre plate, simultaneously. The system consists of a Bioscreen C analyser, which is an incubator and measurement unit, integrated with a PC, software (BioLink v 5.30), printer and a ‘Honeycomb 2’ cuvette multiwell plate. Growth curve data can be analysed within the BioLink software or exported to programs such as Excel.

Broth culture media were dispensed in 270 μl volumes into the wells as directed. Serial dilutions of a filter-sterilised APP stock solution were then dispensed into the same wells, as appropriate. The wells were inoculated with 30 μl of an appropriately diluted overnight broth culture or spore suspension, to give a final inoculum level of cat 110 cfu/ml. The tests were incubated in the Bioscreen C for either 24 h at 30° C., or 72 h at 25° C. (depending on the micro-organism under investigation) with readings taken every 20 minutes after the trays were shaken. After the incubation period was complete the data were exported to Excel for analysis.

2.3.3. Sensitivity Testing for Clostridium Species

Stock solutions of APP215 (0.5, 1 and 2%) were prepared and filter-sterilised. Cooked meat medium (CMM, Oxoid) was prepared by distributing 1 g of the medium to individual test tubes, which were then filled with 8.9 ml water. After autoclaving 100 μl of the APP stock solutions were added, to give final concentrations of 0, 500, 1090 and 2000 ppm APP. The tests were inoculated with 1100 μl of Clostridium spores, to give an inoculum level of ca. 10 cfu/ml. The tubes were overlaid with 3 ml of 2% agar to maintain anaerobicity. The tests were incubated at 37° C., and examined daily for signs of growth (turbidity, gas production).

2.4.1 In vitro Cidal Experiment

100 μl of SDW (sterile deionised water) or APP sample E002012 was added to 890 μl 10 mM HEPES buffer (pH 7). 10 μl of an overnight culture of L. monocytogenes S23 was added to the test, which was incubated at ambient temperature for 2 h. After this time the bacteria were enumerated by viable count. The concentration of the APP sample was 24.7 μg/ml.

2.5 IN vivo (Food) Growth Inhibition Tests

2.5.1 Food Testing in Chicken Soup

A local supermarket brand of cream of chicken soup was used as a model food system. 600 g packs were purchased from the local supermarket. This was a ‘fresh home-made type’ of soup that was stored in the chill cabinet, with a short shelf life. It was chosen because it is a rich, nutritious food system containing a range of food components including meat, dairy and vegetable.

Ingredients were labelled as: chicken stock (water, duck fat, chicken, salt, flavourings, yeast extracts, pork gelatine, glucose syrup, vegetable concentrate, milk protein, sugar, malto-dextrin, vegetable oil, citric acid, lactose, chicken fat), ilk, chicken (9%), cream (8%), onion, potato, vegetable oil, modified maize starch, wheat flour, lemon juice, salt, white pepper. pH 5.912. Protein: 3.3%; carbohydrate: 4.7%; fat: 7.2%; fibre: 0.2%; sodium: 0.1%

For the test the soup was mixed 2:1 in water (to facilitate sampling and mixing) and sterilised by autoclaving at 121° C. for 15 minutes. For each experiment the pH of the soup was recorded; this was between pH 5.79-5.88.

For TCS010412/8 (which was incubated at 8° C.), APP batch number APP20010213 (concentration 169 mg APP/ml) was used to prepare tests containing 0, 2000 or 4000 ppm APP. 5 g of filter-sterilised APP stock solution was added to 94 g of soup, mixed and then 1 ml of a diluted inoculum suspension (cells or spores as appropriate) was added to give a test inoculum level of 10³ cfu/g. The soup mixture was shaken vigorously to ensure homogeneous distribution of the APP and micro-organisms. The tests were tested by viable count and then incubated at 8° C. and tested on a twice-weekly basis.

For TCS010430/20 (incubated at 20° C.) samples were prepared in 18.8 g quantities, to which 1 g of a suitable filter-sterilised APP stock solution was added and an appropriately diluted 0.2 ml inoculum. Great care was again taken to ensure homogeneous distribution of the APP and micro-organisms. This test was sampled by viable count test on a daily basis.

For TCS010521/8 (incubated at 8° C.), samples were prepared in 27.8 g quantities. 1.5 g of an APP stock solution was added, and the tests inoculated with 0.3 ml of a suitably diluted cell or spore suspension to give a final inoculum of ca. 10³ cfu/g. APP was tested at 0, 500 and 1000 ppm, and batch number APP20010213 (at 169 mg/ml) was used. The tests were sampled by viable count and then incubated at 8° C. and tested on a twice-weekly basis.

2.5.2 Test in Apple Juice [TAJ010530/25]

8.9 ml apple juice samples (pH 3.42) were prepared and autoclaved. Batch number APP20010215 (at 138 mg/ml) was used to prepare filter-sterilised stock solutions for 1 ml addition to the juice to achieve 0, 1000, 2000 ppm APP. Tests were inoculated with 100 μl of an appropriately diluted overnight SLM broth of yeast culture or ascospore suspension of Byssochlamys spores prepared earlier. The tests were incubated at 25° C., and examined daily for signs of growth (e.g. turbidity, mycelium development).

2.5.3 Test in Milk [TMGB010605]

8.9 ml samples of UHT milk containing 0.5% glucose and the indicator bromocresol purple (0.01%) were prepared and sterilised by autoclaving. The pH before APP addition was pH 6.65. Appropriate stock solutions of APP batch number APP20010215 (at 138 mg/ml) were prepared and filter sterilised. 1 ml of each was added to the milk to prepare tests containing 0, 1000 and 2000 ppm APP. Tests were then inoculated with 100 μl of diluted overnight broth cultures or spore suspensions to give a final inoculum level of ca. 10³ cfu/ml. These and uninoculated tests were incubated at 20 and 8° C. and examined daily for visible changes compared to uninoculated control.

2.5.4. Test in Cooked Sausage Meat Slurry [TSM010605]

500 g of bologna sausage meat mixture was mixed with 500 g Brain Heart Infusion broth (BHIB) to make a homogeneous slurry. The bologna sausage meat mix comprised (g per kg): lean beef 320; lean pork 202; lard 250; water 40; glucose 5; starch 155; spice mix 3.75; sodium glutamate 0.5; sodium erythorbate 0.545; sodium di-phosphate 3; NaCl 20; sodium nitrite 0.15. 200 g samples of the meat slurry were autoclaved. While the meat was still warm it was re-mixed thoroughly. When the meat had cooled it was inoculated with 2 ml of 10⁵ cfu/ml spore or cell suspension, and re-mixed again. Each inoculated meat sample was divided into 3×50 g samples, and 1 ml of either SDW, 2.5% or 5% APP solution (batch number APP 20010215) was added to prepare meat samples containing 0, 500 and 1000 ppm APP. The meat slurry was again shaken thoroughly to ensure even mixing, an initial viable count was taken and the samples incubated at 8° C. Samples were taken at twice-weekly intervals. The pH of the sausage meat slurry was 6.2.

2.5.5. Test in Cooked Minced Beef Slur [TCM010619]

1 kg of minced beef, purchased from the local butcher, was mixed with 1 litre of BHI. 200 g portion of this meat slurry was autoclaved. When the meat had cooled, the pH was recorded (pH 6.24), and each pot was inoculated with 2.0 ml of 10⁵ spores or cells and the meat re-mixed thoroughly. Each inoculated 200 g pot was distributed as 3×50 g portions. To each pot 1 ml of SDW, 5% or 10% APP (Batch No. APP20010215) was added, and the meat was again mixed thoroughly. An initial viable count was performed, the meat was incubated at 8° C. and sampled twice weekly. The pH of the cooked minced beef slurry was 6.24.

2.5.6. Cooked Minced Chicken Slurry [TCC010704]

1 kg of minced chicken (mainly lean meat) was purchased from the local butcher. This was mixed with 1 litre of BHI, mixed and dispensed as 100 g samples prior to autoclaving. When still warm, the meat was re-mixed thoroughly. When cooked, the meat was inoculated with 1 ml of 10⁵ spores or cells (from an overnight broth), and re-mixed. The samples were then divided into 3×25 g portions, to which 0.5 ml of filter-sterilised APP20010215 was added (as 5% or 10% stock solutions) or water. An initial viable count was taken, and the samples then incubated at 8° C. and tested twice weekly. The pH of the chicken slurry was 6.42.

2.5.7. Uninoculated Test in Raw Meat [TRM010521]

300 g of raw minced beef was purchased from the local butcher. 10 g of either a 2% (20,000 ppm) stock solution of APP2001213 or water was added, and the meat mixed thoroughly. Initial counts were carried out, and then the meat was incubated at 8° C. and sampled for 3 consecutive days. The pH of the raw meat was 5.3.

2.5.8. Inoculated Test in Raw Meat Test [TRM010620]

Fresh beef steak was purchased from the local butcher, and minced on clean machinery. The raw meat (ca. pH 6.2) was divided into 8×90 samples and inoculated-with 10³ cfu/ml of test organisms. The meat was mixed thoroughly using a stomacher. 10 g of SDW or 2% APP (Batch number APP20010215) was added, and the meat was again thoroughly mixed. An initial viable count was taken, the meat was incubated at 8° C., and sampled on the following days using non-selective agar (MPCA) and selective agar: XLD for Salmonella; Oxford selective agar (Listeria monocytogenes); and STAA agar (Brochothrix thermosphacta). The pH of the raw meat was pH 5.3.

3. Results

3.1. In vitro Growth Inhibition Results

3.1.1. Well Diffusion Results

The concentration of the test solution (unknown at time of testing) was 49.3 mg/ml (4.9%). All strains tested were sensitive to this level. They included: Bacillus cereus 204; Clostridium sporogenes Campden; Listeria monocytogenes S23; Micrococcus luteus; Lactobacillus sake A10; Brochothrix thermosphacta CRA7883; Pseudomonas fluorescens 327; Saccharomyces carlsbergensis CRA6413; Saccharomyces cerevisiae ATCC 9763.

3.1.2. Bioscreen Test Results: in vitro Sensitivity of Bacteria and Yeasts

The Bioscreen test results are summarised in Tables 1-4 and FIGS. 3-7. Bioscreen run BS010510 was interrupted by power failure due to electrical storm overnight

3.1.3. Sensitivity of Clostridium Species to APP

No growth inhibition was observed at the highest level tested (2000 ppm APP20010215) against four strains of Cl. sporogenes (strains 1.221; Campden; ABC20; 4.440) and one strain of Cl. tyrobutyricum 2753 grown in cooked meat medium anaerobically at 37° C.

3.2. In vitro Cidal Test Results

After 2 h incubation at ambient temperature in buffer containing 24.7 μg/ml APP, the Listeria counts remained steady, (Table 5).

3.3. In vivo (Food) Growth Inhibition Test Results

3.3.1. Results of Chicken Soup Trials

The results of the chicken soup experiments incubated at 8° C. [TCS010412, TCS010521] and 20° C. [TCS010430] are shown in Tables 6-8, and FIGS. 8-11. The APP had no effect on the colour of the product

3.3.2 Results of Trials in Apple Juice Incubated at Ambient Temperature [TAJ010530]

1000 and 2000 ppm APP controlled the growth of Zygosaccharomyces bailii CRA229 for 34 days. 2000 ppm controlled the growth of S. cerevisiae ATCC9763 for 1 day. No growth inhibition was achieved by 1000 or 2000 ppm APP against Candida parapsilosis 458; Hanseniaspora uvarum CBS5074; Rhodotorula mucilaginosa var mucilaginosa CBS8161; and the heat-resistant fungus Byssochlamys fulva 040021, and B. nivea 163642.

3.3.3 Results of Milk Trials Incubated at 8 and 20° C. [TMGB010605]

At 20° C. results were as follows:

No control observed at 2000 ppm for L. monocytogenes, Br. thermosphacta, Lb. sake.

Control for 2-3 d at 2000 ppm for Ps. fluorescens

Control for 4 d at 1000 ppm, and 6 d at 2000 ppm for B. cereus

At 8° C. results were as follows:

No control observed at 2000 ppm for L. monocytogenes, Lb. sake, B. cereus

Control for 1 d at 2000 ppm for Br. thermosphacta

Control for >7 d at 2000 ppm for Ps. fluorescens

3.3.4. Results of Trial in Cooked Sausage Meat Slurry at 8° C. [TSM010605] (pH 6.2)

Results are summarised in Table 9.

3.3.5. Results of Trial in Cooked Minced Beef Slurry [TCM010619]

Graphs of growth curves are shown in FIG. 12 and summarised in Table 10.

3.3.6. Results of Trial in Cooked Minced Chicken Slur at 8° C. [TCC010704]

Growth curves are shown in FIG. 13, and summarised in Table 11.

3.3.7. Results of Uninoculated Trial in Raw Meat at 8° C. [TRM0105211] (pH 5.31)

Results are shown in Table 12: counts were identical in the control and APP samples. No negative effect of APP on the meat colour was observed.

3.3.8 Results of Inoculated Trial in Raw Meat at 8° C. [TRM010620]

Despite every precaution the TAVC of the meat was high; 10⁶ cfu/g rising to >10⁷ cfu/g by the end of the trial. No significant effect of APP was observed. No negative effect was observed on the meat colour.

### 4.0 CONCLUSIONS

The results of the above tests are summarised in Table 13. The tests indicate that 2000 ppm APP is efficacious against the following micro-organisms: B. cereus, L. monocytogenes, Ps. fluorescens, Salmonella Typhimurium, Lb sake, Y. enterocolitica, Br. thermosphacta, Z bailii and S. cerevisiae.

The observed inhibitory activity is probably bacteriostatic, but in the presence of additional preservative hurdles (particularly low temperature), a cidal effect is possibly achieved with time. Against some bacteria, efficacy of APP was better in a food system compared to in laboratory media, probably because the organism grew less well in the food. The efficacy of APP may vary with different foods due to the differences in their chemical compositions. For example, APP was less efficient in a sausage meat model than in chicken soup.

5.0 Tabulated Data

TABLE 1
Bioscreen results: effective levels of APP against Gram-positive bacteria

| | APP LEVEL (ppm) CAUSING SIGNIFICANT OR TOTAL | | | | | |
|---|---|---|---|---|---|---|
| | INHIBITION FOR TIME/TEMPERATURE INDICATED | | | | | |
| | BS010510 | | BS010411 | | BS010411 | |
| GRAM POSITIVE | 24 h at 30° C. | | 24 h at 30° C. | | 24 h at 30° C. | |
| [Bacillus tested as | [APP20010213] | | [APP20010213] | | [APP20010215] | |
| spores] | Total | Significant | Total | Significant | Total | Significant |
| B. cereus 204 | | | >4000 | 4000 | 4000 | 2000 |
| B. cereus Campden | | | 4000 | 2000 | >4000 | 2000 |
| B. cereus 3.046 | 3000 | 2000 | | | | |
| B. subtilis Campden | 3000 | 2000 | | | | |
| L. monocytogenes 358 | 3000 | | | | | |
| L. monocytogenes 272 | | | 4000 | 2000 | >4000 | 2000 |
| L. monocytogenes S23 | | | 4000 | 2000 | 4000 | 1000 |
| L. monocyt. Scott A | 3000 | 2000 | | | | |
| L. monocyt. F6861 | 3000 | 2000 | | | | |
| Lb. sake A10 | | | 500 | 250 | 500 | 250 |

TABLE 2
Bioscreen results: effective levels of APP against Br. thermosphacta

BS010420 72 h at 25° C.
[APP20010213] APP (ppm)

| | Total | Significant |
|---|---|---|
| Br. thermosphacta | 4000 | 2000 |
| CRA7883 | | |

TABLE 3
Bioscreen results: effective levels of APP against Gram-negative bacteria

| | APP LEVEL (ppm) CAUSING SIGNIFICANT OR TOTAL | | | | | |
|---|---|---|---|---|---|---|
| | INHIBITION FOR TIME/TEMPERATURE INDICATED | | | | | |
| | APP20010213 | | APP 20010213 | | APP 20010215 | |
| | BS010510 | | BS010411 | | BS010411 | |
| GRAM | 24 h/30 | | 24 h/30° C. | | 24 h/30° C. | |
| NEGATIVES | Total | Significant | Total | Significant | Total | Significant |
| Ps. fluorescens | | | 2000 | 1000 | 2000 | 1000 |
| 3756 | | | | | | |
| Ps. fluorescens | 2000 | 1000 | | | | |
| 10460 | | | | | | |
| Ps. fluorescens | 1000 | | | | | |
| 1331 | | | | | | |
| Ps. fluorescens 327 | 1000 | | | | | |
| Ent. aerogenes | 4000 | | | | | |
| 10102 | | | | | | |
| Y. enterocolitica | 2000 | | | | | |
| S16 | | | | | | |
| Salm. | | | 4000 | 2000 | >4000 | 2000 |
| Typhimurium S29 | | | | | | |
| Salmonella sp. S19 | 4000 | | | | | |

TABLE 4
Bioscreen results: effective levels of APP against yeasts

| | APP 213 BS010420 | |
|---|---|---|
| | 72 h/25° C. | |
| YEASTS | Total | Significant |
| S. cerevisiae ATCC9763 | >4000 | Enhanced growth |
| S. carisbergensis CRA6413 | >4000 | Enhanced growth |
| S cerevisiae H78 | >4000 | 4000 |
| S cerevisiae var. paradoxus | >4000 | 2000 |
| H103 | | |

TABLE 5
Results of cidal experiment with APP E002012

| Strain | 0 μg/ml APP | 24.7 μg/ml APP |
|---|---|---|
| L. monocytogenes 523 | 4.3 × 10⁶ | 1.3 × 10⁷ |

TABLE 6
Chicken soup incubated at 8° C. [TCS010412] APP20010213 tested at 0,
2000, 4000 ppm

| Strain | Result |
|---|---|
| B. cereus 204 | Total inhibition by 2000 and 4000 ppm for >60 days. |
| | Counts remain at initial level, i.e. no spore kill. |
| L. monocytogenes 272 | Total inhibition by 4000 ppm for >60 days. |
| | Counts reach <105 with 2000 ppm. |
| | Counts undetectable after 20 days at 4000 ppm |
| | Counts undetectable after ca 50 days at 2000 ppm |
| Ps. fluorescens 3756 | Total inhibition by 2000 and 4000 ppm for >60 days. |
| | Counts undetectable from day 1 in APP samples |

TABLE 7
Chicken soup at 8° C. [TCSM010521]. APP215 tested at 0, 500, 1000 ppm

| Strain | Result |
|---|---|
| B. cereus 204 | Total inhibition by 500 and 1000 ppm for >27 days. |
| | Counts remain at approx initial level, i.e. no spore kill. |
| L. monocytogenes 272 | No total inhibition by 1000 ppm, but slight reduction of |
| | growth rate |
| | No effect with 500 ppm |
| L monocytogenes S23 | No total inhibition by 1000 ppm |
| | Slight reduction of growth rate with 500 and 1000 ppm |
| Ps. fluorescens 3756 | Total inhibition by 1000 ppm for >27 days. |
| | At 1000 ppm counts undetectable >3 d |
| | Little effect with 500 ppm |
| Ps. fluorescens 327 | Total inhibition by 1000 ppm for >27 days. |
| | At 1000 ppm counts undetectable >7 d |
| | Little effect with 500 ppm |
| Salmonella S19 | Total inhibition by 500-1000 ppm for 22 d |
| | Slow growth in controls, reached 10⁶ by 12 d |
| Salm Typhimurium S29 | Total inhibition by 1000 ppm for >27 d |
| | Total inhibition by 500 ppm for 25 d |
| | Slow growth in controls, reached 10⁶ by 14 d |
| S. cerevisiae 9763 | No effect with 500 and 1000 ppm |

TABLE 8
Chicken soup incubated at 20° C. [TCSM010430]. APP20010213 tested at 0,
200, 500, 1000, 2000 ppm

| Strain | Result |
|---|---|
| B. cereus 204 | Total inhibition by 2000 ppm for >42 days. |
| | Extension of lag with 1000 ppm |
| | No significant effect with 500 ppm |
| L. monocytogenes 272 | No total inhibition but reduction of growth rate only by 2000 |
| | ppm |
| | No significant effect by 1000 ppm |
| Ps. fluorescens 3756 | Total inhibition by 2000 ppm for >42 days (counts |
| | undetectable). |
| | No significant effect with 1000 ppm |
| Salmonella Typhimurium | Reduction in final count by 2000 ppm (10⁶ cf 10⁹). After 8 d |
| S29 | counts drop, undetectable by 18 d. |
| | No effect with 1000 ppm |
| S. cerevisiae 9763 | No effect with 2000 ppm |

TABLE 9
Summarised results of cooked sausage meat slurry at 8° C. [TSM010605]

| Strain | Result |
|---|---|
| B. cereus 204 | No total inhibition by 1000 ppm, 10⁶ reached at 7 d |
| | Controls reached 10⁶ by 5 d. |
| L. monocytogenes 272 | No total inhibition by 1000 ppm, 10⁶ reached at 7 d |
| | Controls reached 10⁶ by 7 d |
| Br. thermosphacta | No significant effect with 500 and 100 ppm |
| CRA78884 | |
| Lb. sake A10 | No effect with 500 and 1000 ppm |
| Salm Typhimurium S29 | Total inhibition by 500 for 25 d |
| | Total inhibition by 1000 ppm for >38 d |
| | Slow growth in controls, reached 10⁶ by 19 d |

TABLE 10
Summary of results of trial in minced beef slurry at
8° C. [TCM010619]

| Strain | Result |
|---|---|
| B. cereus 204 | 2000 ppm delayed growth to 10⁶ by 7 d. |
| | 1000 ppm extended lag phase. |
| L. monocytogenes 272 | No significant inhibition by 2000 ppm |
| Br. thermosphacta | No effect with 2000 ppm |
| CRA78883 | |
| Lb. sake A10 | No effect with 2000 ppm |
| Salm Typhimurium S29 | Total inhibition by 2000 ppm for >44 d |
| | Slow growth in controls, reached 10⁶ by 13 d |
| Ps. fluorescens 3756 | Total inhibition by 2000 ppm for 27 d |
| | 1000 ppm delayed growth by 17 d |
| | Controls reached 10⁶ by 15 d |

TABLE 11
Summary of results of trial in minced chicken slurry at 8° C. [TCC010704]

| Strain | Results after 13 d incubation |
|---|---|
| B. cereus 204 | Partial inhibition by 2000 ppm |
| | Controls reached 10⁶ by 5 d |
| L. monocytogenes 272 | Slight inhibition by 2000 ppm only |
| Salm Typhimurium S29 | Total inhibition by 1000 ppm for 25 d |
| | Total inhibition by 2000 ppm for >40 d |
| | Controls not reached 10⁶ by 14 d |
| Ps. fluorescens 327 | No total inhibition by 2000 ppm, 10⁶ reached at 10 d |
| | Controls reached 10⁶ by 3 d |

TABLE 12
Results of raw meat with added at 8° C. [TRM010521]

Viable counts (cfu/g)

| Date | Day | 0 ppm APP | 2000 ppm APP |
|---|---|---|---|
| 21/5 | 0 | 5.6 × 10⁸ | 9.3 × 10⁶ |
| 22/5 | 1 | 2.0 × 10⁸ | 2.2 × 10⁸ |
| 23/5 | 2 | 2.7 × 10⁸ | 1.5 × 10⁸ |
| 24/5 | 3 | 9.7 × 10⁸ | 5.0 × 10⁸ |

TABLE 13
Summary of test results for activity of 2000 ppm APP in food systems

| | Effect on growth of target organism by 2000 ppm APP | | |
|---|---|---|---|
| Food/test systems | Total inhibition | Partial inhibition | No inhibition |
| Lab media at optimum | Lb. sake | B. cereus | Most yeasts |
| temperature | Ps. fluorescens | L. monocytogenes | (enhanced growth) |
| | Y. enterocolitica | Br. thermosphacta | All Clostridium spp |
| | | Salmonella spp | |
| Chicken soup at 8° C. | B. cereus | | S. cerevisiae (1000 |
| pH 5.8 | L. monocytogenes | | ppm) |
| | Ps. fluorescens | | |
| | Salmonella spp | | |
| Chicken soup at 20° C. | B. cereus | L. monocytogenes | S. cerevisiae |
| pH 5.8 | Ps. fluorescens | Salmonella | |
| Apple juice at ambient | — | Z. bailii | Candida |
| pH 3.4 | | S. cerevisiae | parapsilosis |
| | | | H. uvarum |
| | | | Rh. mucilaginosa |
| | | | Byssochlamys fulva |
| Milk at 20° C. | — | Ps. fluorescens | L. monocytogenes |
| pH 6.65 | | B. cereus | Br. thermosphacta |
| | | | Lb. sake |
| Milk at 8° C. | Ps. fluorescens | Br. thermosphacta | L. monocytogenes |
| pH 6.65 | | | Lb. sake |
| | | | B. cereus |
| Cooked sausage meat | S. Typhimurium | B. cereus (1000) | Br. thermosphacta |
| slurry at 8° C. | (1000) | L. monocytog. | (1000) |
| pH 6.2 | | (1000) | Lb. sake (1000) |
| Cooked minced beef | S. Typhimurium | B. cereus | Br. thermosphacta |
| slurry at 8° C. | Ps. fluorescens | | Lb. sake |
| pH 6.24 | | | (L. monocytogenes) |
| Cooked minced chicken | S. Typhimurium | Ps. fluorescens | (L. monocytogenes) |
| slurry at 8° C. | | B. cereus | |
| pH 6.42 | | | |
| Raw uninoculated meat | — | — | TAVC |
| at 8° C. pH 5.3 | | | |
| Raw inoculated meat at | — | | TAVC |
| 8° C. pH 5.3 | | | L. monocytogenes |
| | | | Salmonella |
| | | | Typhimurium |
| | | | Br. thermosphacta |

Various modifications and variations of the described methods and system of the invention will be apparent to those skilled in the art without departing from the scope and spirit of the invention. Modifications of the described modes for carrying out the invention which are obvious to those skilled in the relevant are, or related fields, are thus intended to fall within the scope of the following claims.

The invention will now be further described by the following numbered paragraphs:

1. An antimicrobial composition for use against a micro-organism selected from Listerla, Salmonella, Bacillus, Saccharomyces, Pseudomonas, Clostridium, Lactobacillus, Brochothrix, Micrococcus, Yersinig Enterobacter and Zygosaccharomyces, said composition comprising a cyclic compound having Formula I,

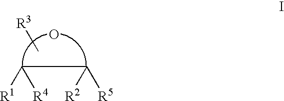

I or a derivative thereof,

wherein R1 and R2 are independently selected from —OH, ═O, and OR′,

wherein R′ is H or —COR″, and R″ is C₁₋₁₀ alkyl;

wherein R3 is a substituent comprising an —OH group;

wherein R4 and R5 are each independently selected from a hydrocarbyl group, H, OH or ═O, or represent a bond with an adjacent atom on the ring of the cyclic compound.

2. A process for preventing and/or inhibiting the growth of, and/or killing, micro-organisms in a material, the process comprising the step of contacting the material with a cyclic compound having Formula I,

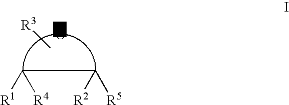

I or a derivative thereof,

wherein R1 and R2 are independently selected from —OH, ═O, and OR′,

wherein R′ is H or —COR″, and R″ is C₁₋₁₀ alkyl;

wherein R3 is a substituent comprising an —OH group;

wherein R4 and R5 are each independently selected from a hydrocarbyl group, H, OH or ═O, or represent a bond with an adjacent atom on the ring of the cyclic compound;

wherein said micro-organism is selected from Listeria, Salmonella, Bacillus, Saccharomyces Pseudomonas, Clostridium, Lactobacillus, Brochothrix, Micrococcus, Yersinia, Enterobacter and Zygosaccharomyces.

3. Use of a compound having Formula I, or a derivative thereof,

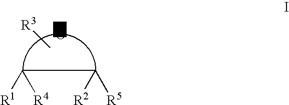

wherein R1 and RZ are independently selected from —OH, ═O, and OR′,

wherein R′ is H or —COW′, and R″ is C₁₋₁₀ alkyl;

wherein R3 is a substituent comprising an —OH group;

wherein R4 and R5 are each independently selected from a hydrocarbyl group, H, OH or ═O, or represent a bond with an adjacent atom on the ring of the cyclic compound;

for preventing and/or inhibiting the growth of, and/or killing, micro-organisms in a material, wherein said micro-organism is selected from Listeria, Salmonella, Bacillus, Saccharomyces, Pseudomonas, Clostridium, Lactobacillus, Brochothrix, Micrococcus, Yersinia, Enterobacter and Zygosaccharomyces.

4. The invention according to any one of the preceding paragraphs wherein said material is a foodstuff or feed.

5. The invention of any one of the preceding paragraphs wherein said cyclic compound is a compound having formula I, or a derivative thereof,

wherein R1 and R2 are independently selected from —OH, ═O;

wherein R3 is a substituent comprising an —OH group;

wherein R4 and R5 are each independently selected from a hydrocarbyl group, H. OH or ═O, or represent a bond with an adjacent atom on the ring of the cyclic compound.

6. The invention of any one of the preceding paragraphs wherein the cyclic compound is a compound having Formula II

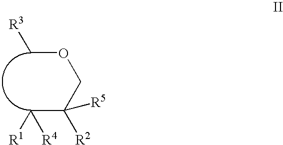

or a derivative thereof, wherein R1, R2, R3, R4, and R5 are as defined in the preceding paragraphs.

7. The invention of any one of the preceding paragraphs wherein the cyclic compound is a compound having Formula III

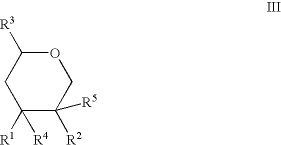

or a derivative thereof; wherein R1, R2, R3, R4, and R5 are as defined in the preceding paragraphs.

8. The invention of any one of the preceding paragraphs wherein the compound is selected from Ascopyrone P, Ascopyrone M, Ascopyrone T, Ascopyrone T1, Ascopyrone T2, Ascopyrone T3, and mixtures thereof.

9. The invention of any one of paragraphs 1 to 3 wherein said cyclic compound is of Formula IV,

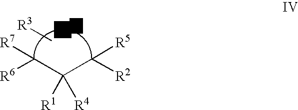

or a derivative thereof, wherein R1 and R2 are independently selected from —OH, ═O, and OR′, wherein R′ is H or —COR″, and R″ is C₁₋₁₀ alkyl;

wherein R3 is a substituent comprising an —OH group;

wherein R4 and R5 are each independently selected from a hydrocarbyl group, H, OH or ═O, or represent a bond with an adjacent atom on the ring of the cyclic compound;

wherein R6 and R7 are each independently selected from H, OH or ═O, or represent a bond with an adjacent atom on the ring of the cyclic compound.

10. The invention of any one of paragraphs 1 to 9 wherein said cyclic compound is of formula V,

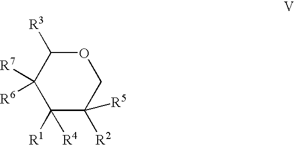

or a derivative thereof, wherein R1, R2, R3, R4, R5, R6 and R′ are as defined in paragraph 9.

11. The invention according to any one of the preceding paragraphs wherein the derivative of the compound of formula I is an ester.

12. The invention according to paragraph 11 wherein the ester is formed from an —OH substituent on the cyclic compound, and wherein said ester is of the formula —(CH₂)ₙ—OC(O)—(CH₂)ₚCH₃, wherein n and p are each independently from 1 to 24.

13. The invention of any one of paragraphs 9 to 12 wherein said cyclic compound is selected from one or more of the following:

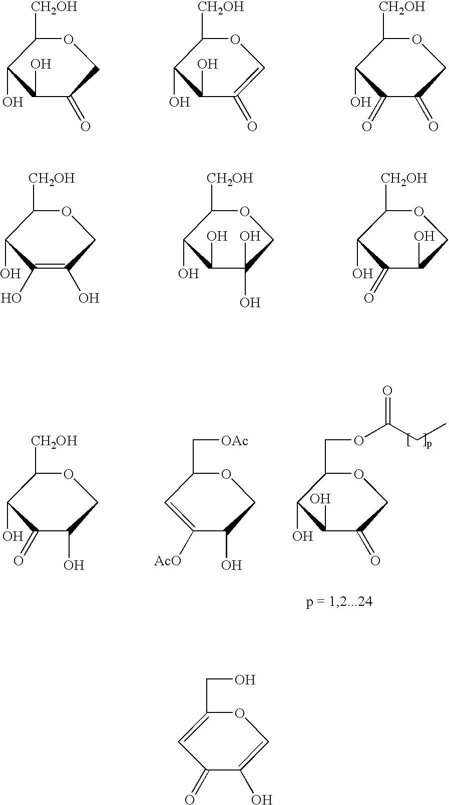

14. The invention of any one of the preceding paragraphs wherein R3 is or comprises a CH₂OH group.

15. The invention of any one of the preceding paragraphs wherein the cyclic compound comprises a five or a six membered ring.

16. The invention according to any preceding paragraph wherein the cyclic compound having formula I has an antimicrobial effect against a micro-organism selected from Listeria monocytogenes, Listeria innocua, Salmonella Typhimurium, Salmonella sp., Bacillus cereus, Bacillus subtilis, Saccharomyces cerevisiae, Saccharomyces cerevisiae var. paradoxes, Saccharomyces carlsbergensis, Pseudomonas fluorescens, Clostridium sporogenes, Lactobacillus sake, Brochothrzx thermosphacta, Micrococcus luteus, Yersinia enterocolitica, Enterobacter aerogenes and Zygosaccharomyces bailii.

17. The invention according to any preceding paragraph wherein said compound of formula I is used in combination-with one or more of an antioxidant, a preservative and/or a chelator.

1. An antimicrobial composition for use against a micro-organism selected from Listerla, Salmonella, Bacillus, Saccharomyces, Pseudomonas, Clostridium, Lactobacillus, Brochothrix, Micrococcus, Yersinig Enterobacter and Zygosaccharomyces, said composition comprising a cyclic compound having Formula I, 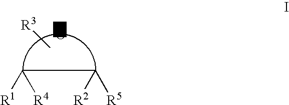
I or a derivative thereof,
wherein R1 and R2 are independently selected from —OH, ═O, and OR′,
wherein R′ is H or —COR″, and R″ is C₁₋₁₀ alkyl;
wherein R3 is a substituent comprising an —OH group;
wherein R4 and R5 are each independently selected from a hydrocarbyl group, H, OH or ═O, or represent a bond with an adjacent atom on the ring of the cyclic compound.

2. A process for preventing and/or inhibiting the growth of, and/or killing, micro-organisms in a material, the process comprising the step of contacting the material with the cyclic compound of claim 1 or a derivative thereof.

3. A method of using the compound of claim 1, or a derivative thereof.

4. The invention according to claim 1 wherein said material is a foodstuff or feed.

5. The invention of claim 1, wherein said cyclic compound is a compound having formula I, or a derivative thereof,
wherein R1 and R2 are independently selected from —OH, ═O;
wherein R3 is a substituent comprising an —OH group;
wherein R4 and R5 are each independently selected from a hydrocarbyl group, H, OH or ═O, or represent a bond with an adjacent atom on the ring of the cyclic compound.

6. The invention of claim 1 wherein the cyclic compound is a compound having Formula II 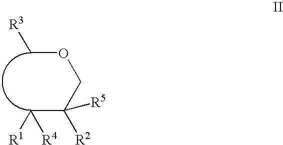
or a derivative thereof, wherein R1, R2, R3, R4, and R5 are as defined in the preceding claims.

7. The invention of claim 1 wherein the cyclic compound is a compound having Formula III 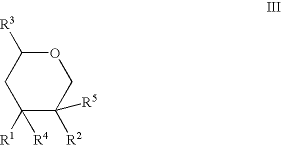
or a derivative thereof; wherein R1, R2, R3, R4, and R5 are as defined in the preceding claims.

8. The invention of claim 1 wherein the compound is selected from Ascopyrone P, Ascopyrone M, Ascopyrone T, Ascopyrone T1, Ascopyrone T2, Ascopyrone T3, and mixtures thereof.

9. The invention of claim 1 wherein said cyclic compound is of Formula IV, 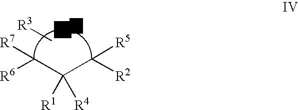
or a derivative thereof, wherein R1 and R2 are independently selected from —OH, ═O, and OR′, wherein R′ is H or —COR″, and R′″ is C₁₋₁₀ alkyl;
wherein R3 is a substituent comprising an —OH group;
wherein R4 and R5 are each independently selected from a hydrocarbyl group, H, OH or ═O, or represent a bond with an adjacent atom on the ring of the cyclic compound;
wherein R6 and R7 are each independently selected from H, OH or ═O, or represent a bond with an adjacent atom on the ring of the cyclic compound.

10. The invention of claim 1 wherein said cyclic compound is of formula V, 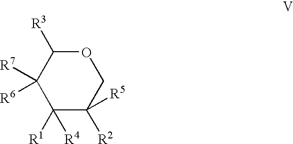
or a derivative thereof, wherein R1,R2, R3, R4, R5, R6 and R′ are as defined in claim 9.

11. The invention according to claim 1 wherein the derivative of the compound of formula I is an ester.

12. The invention according to claim 11 wherein the ester is formed from an —OH substituent on the cyclic compound, and wherein said ester is of the formula —(CH₂)ₙ—OC(O)—(CH₂)ₚCH₃, wherein n and p are each independently from 1 to 24.

13. The invention of claim 9 wherein said cyclic compound is selected from one or more of the following:

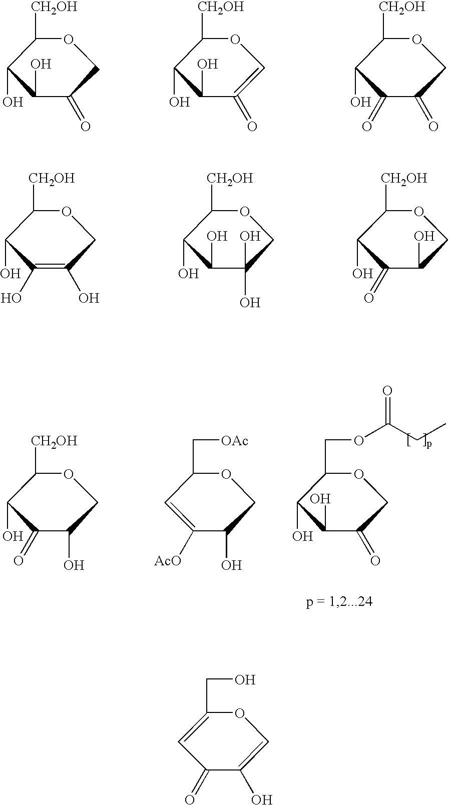

14. The invention of claim 1 wherein R3 is or comprises a CH₂OH group.

15. The invention of claim 1 wherein the cyclic compound comprises a five or a six membered ring.

16. The invention according to claim 1 wherein the cyclic compound having formula I has an antimicrobial effect against a micro-organism selected from Listeria monocytogenes, Listeria innocua, Salmonella Typhimurium, Salmonella sp., Bacillus cereus, Bacillus subtilis, Saccharomyces cerevisiae, Saccharomyces cerevisiae var. paradoxes, Saccharomyces carlsbergensis, Pseudomonas fluorescens, Clostridium sporogenes, Lactobacillus sake, Brochothrzx thermosphacta, Micrococcus luteus, Yersinia enterocolitica, Enterobacter aerogenes and Zygosaccharomyces bailii.

17. The invention according to claim 1 wherein said compound of formula I is used in combination-with one or more of an antioxidant, a preservative and/or a chelator.
